(19) United States
(12) Patent Application Publication
(10) Pub. No.: US 2025/0051287 A1
(43) Pub. Date: Feb. 13, 2025

(54) METHOD FOR PREPARATION OF BENZIMIDAZOLE DERIVATIVES

(71) Applicant: HK INNO.N CORPORATION, Cheongju-si Chungcheongbuk-do (KR)

(72) Inventors: Min Kyoung LEE, Gyeonggi-do (KR); Jae Hong KWEON, Gyeonggi-do (KR); Jungeun LEE, Gyeonggi-do (KR); Yeji PARK, Gyeonggi-do (KR)

(21) Appl. No.: 18/723,780

(22) PCT Filed: Dec. 26, 2022
(86) PCT No.: PCT/KR2022/021316

§ 371 Date: Jun. 24, 2024

(30) Foreign Application Priority Data
Dec. 27, 2021 (KR) 10-2021-0188177

(51) Int. Cl. C07D 235/22

(52) U.S. Cl. CPC C07D 235/22

(57) ABSTRACT

The present invention relates to a method for preparing benzimidazole derivatives and a compound prepared thereby.

### TECHNICAL FIELD

The present invention relates to a method for preparing benzimidazole derivatives and a compound prepared thereby.

### BACKGROUND ART

Benzimidazole is known as a very important pharmacophore in the field of medicine and chemistry. This compound has a fused form of a benzene ring and an imidazole ring in terms of a chemical structure, and has been found to have many pharmaceutical properties. In the 1990s, many benzimidazole derivatives with substituents such as fluoro, propylene, etc., were synthesized and they were shown to have stability, bioavailability and bioactivity.

For example, among the benzimidazole derivatives, oxidazol-1H-benzimidazole is known to have antimicrobial activity and is reported to also have inhibitory activity against fungi, while tetrahydro-imidazol[4,5,1-jk][1,4]-benzodiazepin-2 (1H)-one and N-alkoxy-2-alkyl-benzimidazole have been also developed as HIV inhibitors. In addition, the benzimidazole derivatives with sulfoxide and methylene groups are known to inhibit gastric acid secretion by inhibiting proton pump and have a gastric mucosal protective function. In addition, the benzimidazole derivatives are known to have antiviral and antihypertensive effects.

The benzimidazole derivatives are also very useful in the textile industry, and are known to be mainly used as dye dispersants or softening agents, etc.

As described above, benzimidazole is a pharmacophore having very high applicability in the field of medicine and chemistry, and its synthesis method has been developed by many researchers and has been proposed through various documents.

International Patent Publication WO 2004/054984 describes a preparation method of using 2-amino-3-nitrophenol. However, the preparation method described in said patent uses expensive intermediates and reagents in an amidation reaction, and there is also a difficulty in purification due to the use of a metal catalyst which is difficult to remove. For this reason, the preparation cost is high and the preparation method is not suitable for general mass synthesis processes due to the use of silica gel for the separation of some intermediates. In addition, there is a difficulty in producing only in special facilities due to the use of a metal catalyst with the possibility of fire or explosion.

Furthermore, International Patent Publication WO 2007/072146 describes a method of using carbon monoxide in the process of introducing a carbonyl group for the amidation reaction. However, this reaction requires a separate reaction device and involves a risk factor due to an exposure to carbon monoxide gas during the process. In addition, the preparation cost is high due to the use of expensive reagents and the preparation method is not suitable for general mass synthesis processes due to the use of silica gel for the separation of some intermediates.

Accordingly, the present inventors have confirmed a method capable of industrially mass-producing benzimidazole, which is known as a very important pharmacophore in the field of medicine and chemistry, with a high yield despite the use of commercially available reagents and solvents at low preparation costs, thereby completing the present invention.

### RELATED ART REFERENCE

### Patent Documents

(Patent Document 1) International Patent Publication WO 2004/054984
(Patent Document 2) International Patent Publication WO 2007/072146

### DISCLOSURE OF INVENTION

### Technical Problem

The present invention may provide a method for preparing benzimidazole derivatives.

The present invention may provide a compound prepared by the above preparation method.

### Solution to Problem

This is described in detail as follows. Meanwhile, each description and embodiment disclosed in the present invention may be also applied to other descriptions and embodiments thereof, respectively. In other words, all the combinations of various elements disclosed in the present invention fall within the scope of the present invention. Also, it cannot be seen that the scope of the present invention is limited to the specific description described below.

The present invention may provide a method for preparing benzimidazole derivatives, the method including:
1) preparing a compound represented by formula 2 below by reacting a compound represented by formula 3 below with a monovalent copper catalyst in the presence of a ligand represented by formula 4 below or a stereoisomer thereof;
2) preparing a compound represented by formula 5 below by reacting a compound represented by formula 2 below; and
3) preparing a compound represented by formula 1 below by reacting a compound represented by formula 5 below, acetyl chloride, and a compound represented by formula 6 below in the presence of a base.

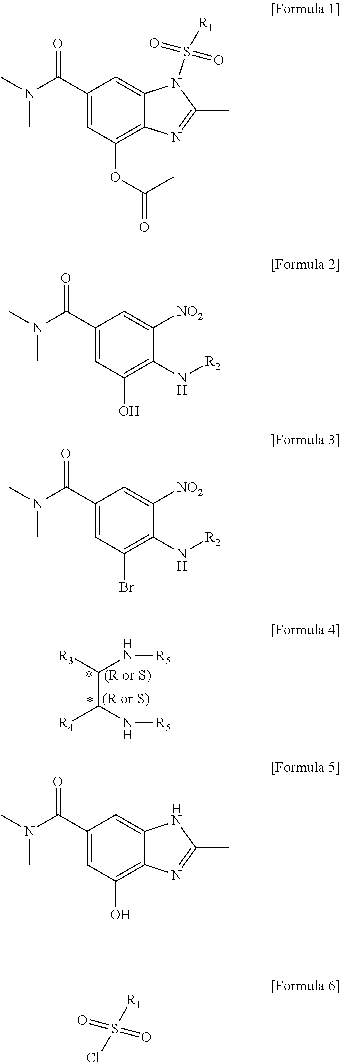

wherein,
R₁ may be C₁₋₄ alkyl or phenyl, in which at least one H of the C₁₋₄ alkyl and phenyl may be substituted with halogen or C₁₋₄ alkyl,
R₂ may be H, C₁₋₄ alkyl or acetyl, in which at least one H of the acetyl may be substituted with halogen,
R₃ and R₄ may be each independently selected from the group consisting of H, C₁₋₄ alkyl and C₃₋₇ cycloalkyl, or together form a 3- to 7-membered alicyclic ring, in which at least one H of the C₁₋₄ alkyl, C₃₋₇ Cycloalkyl and the formed 3- to 7-membered alicyclic ring may be substituted with halogen, and
R₅ may be H, C₁₋₄ alkyl or acetyl.

The method for preparing benzimidazole derivatives may further include:

4) preparing a compound represented by formula 1-1 below by adjusting a pH of the compound represented by above formula 1,

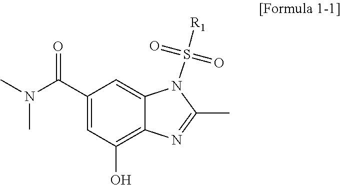

wherein,
R₁ may be the same as in above formula 1.

In one embodiment, R₁ may be methyl, trifluoromethyl or tolyl.

The present invention may provide a method for preparing benzimidazole derivatives, the method including:
1) preparing a compound represented by formula 2 below by reacting a compound represented by formula 3 below with a monovalent copper catalyst in the presence of a ligand represented by formula 4 below or a stereoisomer thereof;
2) preparing a compound represented by formula 5 below by reacting a compound represented by formula 2 below;
3) preparing a compound represented by formula 1 below by reacting a compound represented by formula 5 below, acetyl chloride, and a compound represented by formula 6 below in the presence of a base; and
4) preparing a compound represented by formula 1-1 below by adjusting a pH of the compound represented by formula 1 below,

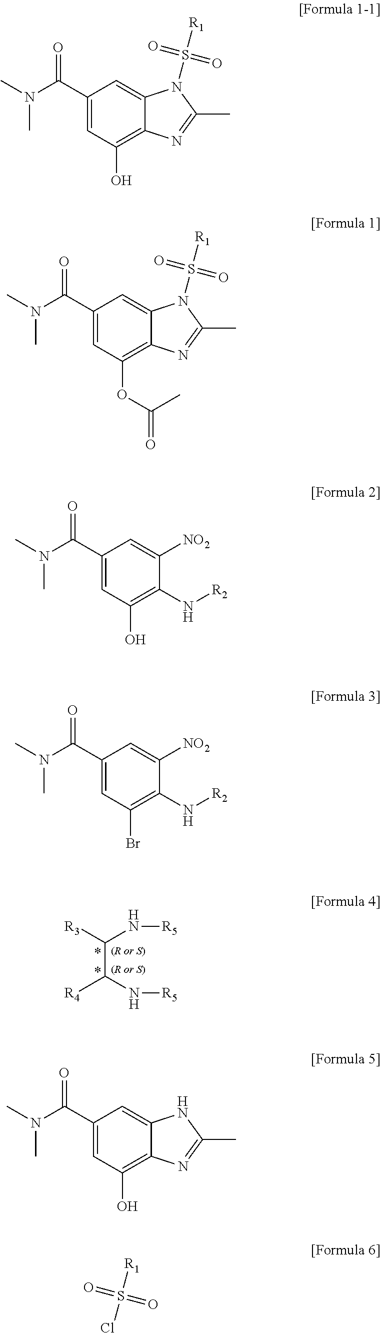

wherein,
R₁ may be C₁₋₄ alkyl or phenyl, in which at least one H of the C₁₋₄ alkyl and phenyl may be substituted with halogen or C₁₋₄ alkyl,
R₂ may be H, C₁₋₄ alkyl or acetyl, in which at least one H of the acetyl may be substituted with halogen,
R₃ and R₄ may be each independently selected from the group consisting of H, C₁₋₄ alkyl and C₃₋₇ cycloalkyl, or together form a 3- to 7-membered alicyclic ring, in which at least one H of the C₁₋₄ alkyl, C₃₋₇ Cycloalkyl and the formed 3- to 7-membered alicyclic ring may be substituted with halogen, and
R₅ may be H, C₁₋₄ alkyl or acetyl.

In one embodiment, in above formula,
R₁ may be C₁₋₄ alkyl or phenyl, in which at least one H of the C₁₋₄ alkyl may be substituted with halogen, and at least one H of the phenyl may be substituted with C₁₋₄ alkyl,
R₂ may be H or acetyl, in which at least one H of the acetyl may be substituted with halogen,
R₃ and R₄ may be C₁₋₄ alkyl, or together form a 3- to 7-membered alicyclic ring, and
R₅ may be H or C₁₋₄ alkyl.

In one embodiment, in above formula,
R₁ may be C₁₋₄ alkyl or phenyl, in which at least one H of the C₁₋₄ alkyl may be substituted with fluoro (F), and at least one H of the phenyl may be substituted with methyl,
R₂ may be H or acetyl, in which at least one H of the acetyl may be substituted with fluoro (F),
R₃ and R₄ may be C₁₋₄ alkyl, or together form a 3- to 7-membered alicyclic ring, and
R₅ may be H or C₁₋₄ alkyl.

In one embodiment, in above formula,
R₁ may be methyl, trifluoromethyl or tolyl,
R₂ may be H, acetyl or trifluoroacetyl,
R₃ and R₄ may be methyl, or together form a 6-membered alicyclic ring, and
R₅ may be H or methyl.

In the present invention, the compound represented by above formula 4 may contain one or more stereocenters, and thus may be present as a mixture of enantiomers including a racemate, a single enantiomer, a mixture of diastereomers or as a single diastereomer.

The method for preparing benzimidazole derivatives according to the present invention may include: 1) preparing a compound represented by above formula 2 by reacting a compound represented by above formula 3 with a monovalent copper catalyst in the presence of a ligand represented by above formula 4 or a stereoisomer thereof.

The monovalent copper catalyst of above 1) may be one selected from the group consisting of cuprous chloride, copper bromide, copper iodide and cuprous oxide, and specifically may be copper bromide, but is not limited thereto.

A molar ratio of the compound represented by above formula 3, the monovalent copper catalyst, and the ligand represented by above formula 4 or a stereoisomer thereof in above 1) may be 10:2:4 to 10:0.5:0.5, but is not limited thereto.

Above 1) may be performed in at least one solvent selected from the group consisting of water, acetonitrile, tetrahydrofuran, dimethylsulfoxide, dimethylformamide and 1,4-dioxane, and may be specifically performed in water and dimethylsulfoxide, but is not limited thereto.

Above 1) may be performed by adjusting a temperature between 37° C. and 60° C. and stirring for 10 to 60 minutes, but is not limited thereto.

The method for preparing benzimidazole derivatives according to the present invention may include: 2) preparing a compound represented by above formula 5 by reacting a compound represented by above formula 2.

Above 2) may be to prepare the compound represented by above formula 5 by subjecting the compound represented by above formula 2 to a reductive cyclization reaction.

The reductive cyclization of above 2) may be performed by adding a reducing agent or a reducing agent and an acetyl source.

The reductive cyclization of above 2) may include a nitro reduction using a reducing agent or a nitro reduction using a reducing agent and cyclization with an acetyl source.

The reducing agent in above 2) may be at least one selected from the group consisting of H₂/10% palladium carbon, reduced iron, and sodium dithionate, but is not limited thereto.

The acetyl source in above 2) may be at least one selected from the group consisting of triethyl orthoacetate and acetyl acetone, but is not limited thereto.

Above 2) may be performed by reacting the compound represented by above formula 2 with H₂/10% palladium carbon and triethyl orthoacetate; reduced iron and triethyl orthoacetate; or sodium dithionate, but is not limited thereto.

The method for preparing benzimidazole derivatives according to the present invention may include: 3) preparing a compound represented by above formula 1 by reacting the compound represented by above formula 5, acetyl chloride, and the compound represented by above formula 6 in a presence of a base.

The base of above 3) may be at least one selected from the group consisting of potassium carbonate, sodium carbonate, cesium carbonate, aqueous ammonia and pyridine, and may be specifically potassium carbonate and pyridine, but is not limited thereto.

A molar ratio of the compound represented by above formula 5 and the compound represented by above formula 6 in above 3) may be 1:1 to 1:2.5, but is not limited thereto.

A molar ratio of the acetyl chloride and the base in above 3) may be 1:2 to 1:4, but is not limited thereto.

A molar ratio of the compound represented by above formula 5 and the acetyl chloride in above 3) may be 1:1 to 1:1.5, but is not limited thereto.

A molar ratio of the compound represented by above formula 6 and the base in above 3) may be 1:1 to 1:4, but is not limited thereto.

Above 3) may be performed in at least one solvent selected from the group consisting of acetone, methyl ethyl ketone, ethyl acetate, methylene chloride, chloroform and acetonitrile, and may be specifically performed in methylene chloride, but is not limited thereto.

Above 3) may include:
a) adding the compound represented by above formula 5 and acetyl chloride to a solvent; and
b) adding the compound represented by above formula 6 in the presence of a base.

The compound represented by above formula 5 in above a) may be added in an amount of 5% (w/v) to 33% (w/v) relative to the solvent, but is not limited thereto.

Above a) may be performed by adjusting a temperature between 3° C. and 40° C. and stirring for 30 to 120 minutes, but is not limited thereto.

Above b) may be performed by adjusting a temperature between 27° C. and 40° C. and stirring for 10 to 180 minutes, but is not limited thereto.

The method for preparing benzimidazole derivatives according to the present invention may include: 4) preparing a compound represented by above formula 1-1 by adjusting a pH of the compound represented by above formula 1.

Above 4) may be to adjust the pH between 8 and 12, but is not limited thereto.

Above 4) may be performed by adjusting a temperature between 3° C. and 10° C., but is not limited thereto.

The method for preparing benzimidazole derivatives according to the present invention may be to perform above 3) and 4) in situ.

The present invention may provide a compound represented by formula 1 below:

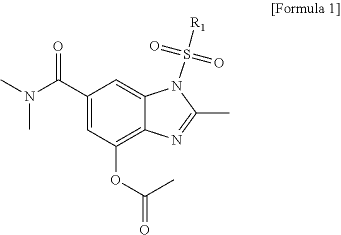

wherein,
R₁ is C₁₋₄ alkyl or phenyl, in which at least one H of the C₁₋₄ alkyl and phenyl may be substituted with halogen or C₁₋₄ alkyl.

In one embodiment, in above formula,
R₁ may be C₁₋₄ alkyl or phenyl, in which at least one H of the C₁₋₄ alkyl may be substituted with halogen, and at least one H of the phenyl may be substituted with C₁₋₄ alkyl.

In one embodiment, in above formula,
R₁ may be C₁₋₄ alkyl or phenyl, in which at least one H of the C₁₋₄ alkyl may be substituted with fluoro (F), and at least one H of the phenyl may be substituted with methyl.

In one embodiment, R₁ may be methyl, trifluoromethyl or tolyl.

The compound represented by above formula 1 may be selected from the group consisting of:
6-(dimethylcarbamoyl)-2-methyl-1-tosyl-1H-benzo[d]imidazol-4-yl acetate;
6-(dimethylcarbamoyl)-2-methyl-1-((trifluoromethyl) sulfonyl)-1H-benzo[d]imidazol-4-yl acetate; and
6-(dimethylcarbamoyl)-2-methyl-1-methylsulfonyl-1H-benzo[d]imidazol-4-yl acetate.

The present invention may provide methods of the following (1) to (28):

(1) A method for preparing benzimidazole derivatives, the method including:
1) preparing a compound represented by formula 2 below by reacting a compound represented by formula 3 below with a monovalent copper catalyst in the presence of a ligand represented by formula 4 below or a stereoisomer thereof;
2) preparing a compound represented by formula 5 below by reacting a compound represented by formula 2 below; and
3) preparing a compound represented by formula 1 below by reacting a compound represented by formula 5 below, acetyl chloride, and a compound represented by formula 6 below in the presence of a base,

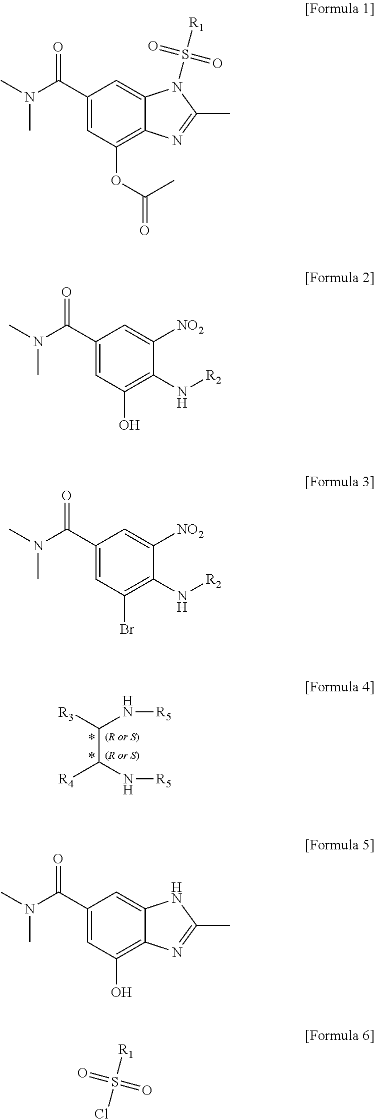

wherein,
R₁ may be C₁₋₄ alkyl or phenyl, in which at least one H of the C₁₋₄ alkyl and phenyl may be substituted with halogen or C₁₋₄ alkyl,
R₂ may be H, C₁₋₄ alkyl or acetyl, in which at least one H of the acetyl may be substituted with halogen,
R₃ and R₄ may be each independently selected from the group consisting of H, C₁₋₄ alkyl and C₃₋₇ cycloalkyl, or together form a 3- to 7-membered alicyclic ring, in which at least one H of the C₁₋₄ alkyl, C₃₋₇ cycloalkyl and the formed 3- to 7-membered alicyclic ring may be substituted with halogen, and
R₅ may be H, C₁₋₄ alkyl or acetyl.

(2) The method of above (1) may further include:
4) preparing a compound represented by formula 1-1 below by adjusting a pH of the compound represented by above formula 1:

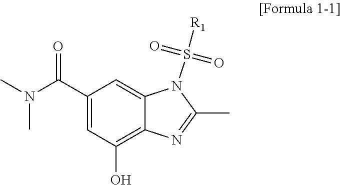

wherein,
R₁ may be the same as in above formula I.

(3) A method for preparing benzimidazole derivatives, the method including:
1) preparing a compound represented by formula 2 below by reacting a compound represented by formula 3 below with a monovalent copper catalyst in the presence of a ligand represented by formula 4 below or a stereoisomer thereof;
2) preparing a compound represented by formula 5 below by reacting a compound represented by formula 2 below;
3) preparing a compound represented by formula 1 below by reacting a compound represented by formula 5 below, acetyl chloride, and a compound represented by formula 6 below in the presence of a base; and
4) preparing a compound represented by formula 1-1 below by adjusting a pH of the compound represented by formula 1 below,

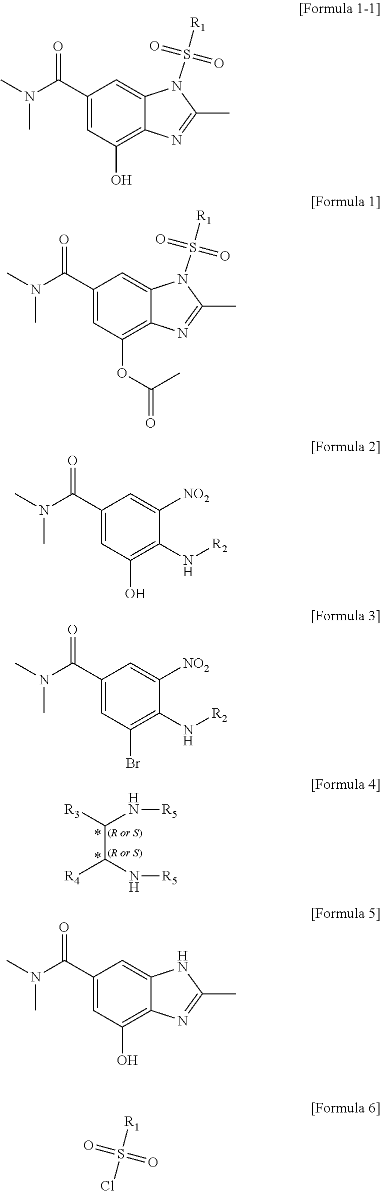

wherein,
R₁ may be C₁₋₄ alkyl or phenyl, in which at least one H of the C₁₋₄ alkyl and phenyl may be substituted with halogen or C₁₋₄ alkyl,
R₂ may be H, C₁₋₄ alkyl or acetyl, in which at least one H of the acetyl may be substituted with halogen,
R₃ and R₄ may be each independently selected from the group consisting of H, C₁₋₄ alkyl and C₃₋₇ cycloalkyl, or together form a 3- to 7-membered alicyclic ring, in which at least one H of the C₁₋₄ alkyl, C₃₋₇ cycloalkyl and the formed 3- to 7-membered alicyclic ring may be substituted with halogen, and
R₅ may be H, C₁₋₄ alkyl or acetyl.

(4) The method of above (1), (2) or (3), wherein,
R₁ may be C₁₋₄ alkyl or phenyl, in which at least one H of the C₁₋₄ alkyl may be substituted with halogen, and at least one H of the phenyl may be substituted with C₁₋₄ alkyl,
R₂ may be H or acetyl, in which at least one H of the acetyl may be substituted with halogen,
R₃ and R₄ may be C₁₋₄ alkyl, or together form a 3- to 7-membered alicyclic ring, and R₅ may be H or C₁₋₄ alkyl.

(5) The method of above (1), (2), (3) or (4), wherein,
R₁ may be C₁₋₄ alkyl or phenyl, in which at least one H of the C₁₋₄ alkyl may be substituted with fluoro (F), and at least one H of the phenyl may be substituted with methyl,
R₂ may be H or acetyl, in which at least one H of the acetyl may be substituted with fluoro (F),
R₃ and R₄ may be C₁₋₄ alkyl, or together form a 3- to 7-membered alicyclic ring, and
R₅ may be H or C₁₋₄ alkyl.

(6) The method of above (1), (2), (3), (4) or (5), wherein,
R₁ may be methyl, trifluoromethyl or tolyl,
R₂ may be H, acetyl or trifluoroacetyl,
R₃ and R₄ may be methyl, or together form a 6-membered alicyclic ring, and
R₅ may be H or methyl.

(7) The method of above (1), (2), (3) or (4), wherein,
R₁ may be methyl, trifluoromethyl or tolyl.

(8) The method of above (1), (2), (3), (4), (5), (6) or (7), wherein the monovalent copper catalyst of above 1) may be one selected from the group consisting of cuprous chloride, copper bromide, copper iodide and cuprous oxide.

(9) The method of above (1), (2), (3), (4), (5), (6), (7) or (8), wherein a molar ratio of the compound represented by above formula 3, the monovalent copper catalyst, and the ligand represented by above formula 4 or the stereoisomer thereof in above 1) may be 10:2:4 to 10:0.5:0.5.

(10) The method of above (1), (2), (3), (4), (5), (6), (7), (8) or (9), wherein above 1) may be performed in at least one solvent selected from the group consisting of water, acetonitrile, tetrahydrofuran, dimethylsulfoxide, dimethylformamide and 1,4-dioxane.

(11) The method of above (1), (2), (3), (4), (5), (6), (7), (8), (9) or (10), wherein above 1) may be performed by adjusting a temperature between 37° C. and 60° C. and stirring for 10 to 60 minutes.

(12) The method of above (1), (2), (3), (4), (5), (6), (7), (8), (9), (10) or (11), wherein above 2) may be to prepare the compound represented by above formula 5 by subjecting the compound represented by above formula 2 to a reductive cyclization reaction.

(13) The method of above (12), wherein the reductive cyclization of above 2) may be performed by adding a reducing agent or a reducing agent and an acetyl source.

(14) The method of above (13), wherein the reducing agent may be at least one selected from the group consisting of H₂/10% palladium carbon, reduced iron and sodium dithionate.

(15) The method of above (13) or (14), wherein the acetyl source may be at least one selected from the group consisting of triethyl orthoacetate and acetyl acetone.

(16) The method of above (1), (2), (3), (4), (5), (6), (7), (8), (9), (10), (11), (12), (13), (14) or (15), wherein the base of above 3) may be at least one selected from the group consisting of potassium carbonate, sodium carbonate, cesium carbonate, aqueous ammonia and pyridine.

(17) The method of above (1), (2), (3), (4), (5), (6), (7), (8), (9), (10), (11), (12), (13), (14), (15) or (16), wherein a molar ratio of the compound represented by above formula 5 and the compound represented by above formula 6 in above 3) may be 1:1 to 1:2.5.

(18) The method of above (1), (2), (3), (4), (5), (6), (7), (8), (9), (10), (11), (12), (13), (14), (15), (16) or (17), wherein a molar ratio of the acetyl chloride and the base in above 3) may be 1:2 to 1:4.

(19) The method of above (1), (2), (3), (4), (5), (6), (7), (8), (9), (10), (11), (12), (13), (14), (15), (16), (17) or (18), wherein a molar ratio of the compound represented by above formula 5 and the acetyl chloride in above 3) may be 1:1 to 1:1.5.

(20) The method of above (1), (2), (3), (4), (5), (6), (7), (8), (9), (10), (11), (12), (13), (14), (15), (16), (17), (18) or (19), wherein a molar ratio of the compound represented by above formula 6 and the base in above 3) may be 1:1 to 1:4.

(21) The method of above (1), (2), (3), (4), (5), (6), (7), (8), (9), (10), (11), (12), (13), (14), (15), (16), (17), (18), (19) or (20), wherein above 3) may be performed in at least one solvent selected from the group consisting of acetone, methyl ethyl ketone, ethyl acetate, methylene chloride, chloroform and acetonitrile.

(22) The method of above (1), (2), (3), (4), (5), (6), (7), (8), (9), (10), (11), (12), (13), (14), (15), (16), (17), (18), (19), (20) or (21), wherein above 3) may include:
a) adding the compound represented by above formula 5 and acetyl chloride to a solvent; and
b) adding the compound represented by above formula 6 in the presence of a base.

(23) The method of above (22), wherein the compound represented by above formula 5 in above a) may be added in an amount of 5% (w/v) to 33% (w/v) relative to the solvent.

(24) The method of above (22) or (23), wherein above a) may be performed by adjusting a temperature between 3° C. and 40° C. and stirring for 30 to 120 minutes.

(25) The method of above (22), (23) or (24), wherein above b) may be performed by adjusting a temperature between 27° C. and 40° C. and stirring for 10 to 180 minutes.

(26) The method of above (2), (3), (4), (5), (6), (7), (8), (9), (10), (11), (12), (13), (14), (15), (16), (17), (18), (19), (20), (21), (22), (23), (24) or (25), wherein above 4) may be to adjust the pH between 8 and 12.

(27) The method of above (2), (3), (4), (5), (6), (7), (8), (9), (10), (11), (12), (13), (14), (15), (16), (17), (18), (19), (20), (21), (22), (23), (24), (25) or (26), wherein above 4) may be performed by adjusting a temperature between 3° C. and 10° C.

(28) The method of above (2), (3), (4), (5), (6), (7), (8), (9), (10), (11), (12), (13), (14), (15), (16), (17), (18), (19), (20), (21), (22), (23), (24), (25), (26) or (27), wherein above 3) and 4) are performed in situ.

The present invention may provide compounds of the following (1) to (5):

(1) A compound represented by formula 1 below:

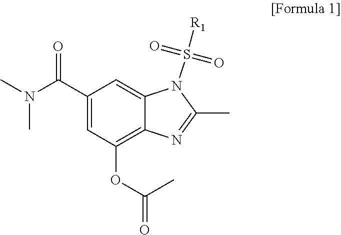

wherein,
R₁ is C₁₋₄ alkyl or phenyl, wherein at least one H of the C₁₋₄ alkyl and phenyl may be substituted with halogen or C₁₋₄ alkyl.

(2) The compound of above (1), wherein R₁ may be C₁₋₄ alkyl or phenyl, in which at least one H of the C₁₋₄ alkyl may be substituted with halogen, and at least one H of the phenyl may be substituted with C₁₋₄ alkyl.

(3) The compound of above (1) or (2), wherein R₁ may be C₁₋₄ alkyl or phenyl, in which at least one H of the C₁₋₄ alkyl may be substituted with fluoro (F), and at least one H of the phenyl may be substituted with methyl.

(4) The compound of above (1), (2) or (3), wherein R₁ may be methyl, trifluoromethyl or tolyl.

(5) The compound of above (1), (2), (3) or (4), wherein the compound may be selected from the group consisting of:
6-(dimethylcarbamoyl)-2-methyl-1-tosyl-1H-benzo[d]imidazol-4-yl acetate;
6-(dimethylcarbamoyl)-2-methyl-1-((trifluoromethyl) sulfonyl)-1H-benzo[d]imidazol-4-yl acetate; and
6-(dimethylcarbamoyl)-2-methyl-1-methylsulfonyl-1H-benzo[d]imidazol-4-yl acetate.

### Advantageous Effects of Invention

The method for preparing benzimidazole derivatives according to the present invention has a low preparation cost, does not require a chromatography process in a preparation process, and does not use high-risk and high-cost reagents, and thus can be advantageous for mass production and can be prepared with high yield.

In addition, a compound prepared by the above preparation method can be used to effectively prepare other compounds having a benzimidazole structure, and in particular can be used as an intermediate for the preparation of compounds usable as antibacterial agents, anti-ulcer agents, and anti-inflammatory agents.

### BEST MODE FOR CARRYING OUT THE INVENTION

Hereinafter, the present invention will be described in more detail through exemplary embodiments. These exemplary embodiments are provided only for the purpose of illustrating the present invention, and thus it will be apparent to those skilled in the art that the scope of the present invention is not limited thereto.

### Example 1. Step 1

### Example 1-1. Preparation of 4-acetamido-3-hydroxy-N,N-dimethyl-5-nitrobenzamide

The 1034.8 g of purified water, 701.9 g of dimethyl sulfoxide and 144.4 g of potassium carbonate were sequentially added to a reactor. The resulting solution was stirred for 30 minutes while an internal temperature was raised to 37° C. The 3.8 g of copper (I) bromide, 7.4 g of trans-N,N-dimethylcyclohexan-1,2-diamine and 344.9 g of purified water were added and stirred while maintaining the internal temperature of 37° C. Then, a solution obtained by dissolving 172.5 g of 4-acetamido-3-bromo-N,N-dimethyl-5-nitrobenzamide in 38.5 g of dimethyl sulfoxide was added to the reactor. The reaction solution was stirred for 60 minutes while maintaining the same at 37° C., so as to complete the reaction. The 345.0 g of distilled water was added and an internal temperature was cooled to 10-15° C. by using an ice bath, after which the resulting mixture was stirred for 12 hours and crystallized. The resulting crystals were filtered and the filtrate was washed with purified water. The obtained solid was vacuum dried at 40° C. to yield 128.4 g of 4-acetamido-3-hydroxy-N,N-dimethyl-5-nitrobenzamide (yield: 92%).

¹H-NMR (400 MHZ, DMSO-d₆): 10.9 (s, 1H), 9.8 (s, 1H), 7.3 (d, 1H), 7.2 (d, 1H), 3.0 (s, 6H), 2.0 (s, 3H)

### Example 1-2. Preparation of 4-amino-3-hydroxy-N,N-dimethyl-5-nitrobenzamide

The 899.8 g of purified water, 610.3 g of dimethyl sulfoxide and 125.6 g of potassium carbonate were sequentially added to a reactor. The resulting solution was stirred for 30 minutes while an internal temperature was raised to 60° C. The 15.5 g of copper (I) bromide, 6.4 g of trans-N,N-dimethylcyclohexan-1,2-diamine and 299.9 g of purified water were added and stirred while maintaining the internal temperature of 60° C. Then, a solution obtained by dissolving 155.2 g of 4-amino-3-bromo-N,N-dimethyl-5-nitrobenzamide in 33.5 g of dimethyl sulfoxide was added to the reactor. The reaction solution was stirred for 10 minutes while maintaining the same at 60° C., so as to complete the reaction. The 300.0 g of distilled bath, after which the resulting mixture was stirred for 12 hours and crystallized. The crystals were filtered and the filtrate was washed with purified water. The obtained solid was vacuum dried at 40° C. to yield 109.2 g of 4-amino-3-hydroxy-N,N-dimethyl-5-nitrobenzamide (yield: 90%).

¹H-NMR (400 MHZ, DMSO-d₆): 10.7 (s, 1H), 7.6 (d, 1H), 7.1 (s, 2H), 7.0 (s, 1H), 3.0 (s, 6H)

### Example 1-3. Preparation of 4-trifluoroacetamido-3-hydroxy-N,N-dimethyl-5-nitrobenzamide

The 1010.8 g of purified water, 685.6 g of dimethyl sulfoxide and 141.1 g of potassium carbonate were sequentially added to a reactor. The resulting solution was stirred for 90 minutes while an internal temperature was raised to 45° C. The 3.7 g of copper (I) bromide, 7.2 g of trans-N,N-dimethylcyclohexan-1,2-diamine and 336.9 g of purified water were added and stirred while maintaining the internal temperature of 45° C. Then, a solution obtained by dissolving 163.0 g of 4-trifluoroacetamido-3-bromo-N,N-dimethyl-5-nitrobenzamide in 37.6 g of dimethyl sulfoxide was added to the reactor. The reaction solution was stirred for 60 minutes while maintaining the same at 45° C., so as to complete the reaction. The 337.0 g of distilled water was added and an internal temperature was cooled to 10-15° C. by using an ice bath, after which the resulting mixture was stirred for 12 hours and crystallized. The resulting crystals were filtered and the filtrate was washed with purified water. The obtained solid was vacuum dried at 40° C. to yield 117.2 g of 4-trifluoroacetamido-3-hydroxy-N,N-dimethyl-5-nitrobenzamide (yield: 86%).

¹H-NMR (400 MHZ, DMSO-d₆): 10.9 (s, 1H), 9.8 (s, 1H), 7.3 (d, 1H), 7.2 (d, 1H), 3.0 (s, 6H)

### Example 1-4. Preparation of 4-acetamido-3-hydroxy-N,N-dimethyl-5-nitrobenzamide

The 1034.8 g of purified water, 701.9 g of dimethyl sulfoxide and 144.4 g of potassium carbonate were sequentially added to a reactor. The resulting solution was stirred for 30 minutes while an internal temperature was raised to 37° C. The 3.8 g of copper (I) bromide, 5.9 g of trans-1,2-diaminocyclohexane and 344.9 g of purified water were added and stirred while maintaining the internal temperature of 37° C. Then, a solution obtained by dissolving 172.5 g of 4-acetamido-3-bromo-N,N-dimethyl-5-nitrobenzamide in 38.5 g of dimethyl sulfoxide was added to the reactor. The reaction solution was stirred for 60 minutes while maintaining the same at 37° C., so as to complete the reaction. The 345.0 g of distilled bath, after which the resulting mixture was stirred for 12 hours and crystallized. The resulting crystals were filtered and the filtrate was washed with purified water. The obtained solid was vacuum dried at 40° C. to yield 125.6 g of 4-acetamido-3-hydroxy-N,N-dimethyl-5-nitrobenzamide (yield: 90%).

¹H-NMR (400 MHZ, DMSO-d₆): 10.9 (s, 1H), 9.8 (s, 1H), 7.3 (d, 1H), 7.2 (d, 1H), 3.0 (s, 6H), 2.0 (s, 3H)

### Example 1-5. Preparation of 4-amido-3-hydroxy-N,N-dimethyl-5-nitrobenzamide

The 899.8 g of purified water, 610.3 g of dimethyl sulfoxide and 125.6 g of potassium carbonate were sequentially added to a reactor. The resulting solution was stirred for 30 minutes while an internal temperature was raised to 60° C. The 15.5 g of copper (I) bromide, 5.1 g of trans-1,2-diaminocyclohexane and 299.9 g of purified water were added and stirred while maintaining the internal temperature of 60° C. Then, a solution obtained by dissolving 155.2 g of 4-amino-3-bromo-N,N-dimethyl-5-nitrobenzamide in 33.5 g of dimethyl sulfoxide was added to the reactor. The reaction solution was stirred for 10 minutes while maintaining the same at 60° C., so as to complete the reaction. The 300.0 g of distilled water was added and an internal temperature was cooled to 10-15° C. by using an ice bath, after which the resulting mixture was stirred for 12 hours and crystallized. The crystals were filtered and the filtrate was washed with purified water. The obtained solid was vacuum dried at 40° C. to yield 104.3 g of 4-amino-3-hydroxy-N,N-dimethyl-5-nitrobenzamide (yield: 86%).

¹H-NMR (400 MHZ, DMSO-d₆): 10.7 (s, 1H), 7.6 (d, 1H), 7.1 (s, 2H), 7.0 (s, 1H), 3.0 (s, 6H)

### Example 1-6. Preparation of 4-trifluoroacetamido-3-hydroxy-N,N-dimethyl-5-nitrobenzamide

The 1010.8 g of purified water, 685.6 g of dimethyl sulfoxide and 141.1 g of potassium carbonate were sequentially added to a reactor. The resulting solution was stirred for 90 minutes while an internal temperature was raised to 45° C. The 3.7 g of copper (I) bromide, 5.8 g of trans-1,2-diaminocyclohexane and 336.9 g of purified water were added and stirred while maintaining the internal temperature of 45° C. Then, a solution obtained by dissolving 163.0 g of 4-trifluoroacetamido-3-bromo-N,N-dimethyl-5-nitrobenzamide in 37.6 g of dimethyl sulfoxide was added to the reactor. The reaction solution was stirred for 60 minutes while maintaining the same at 45° C., so as to complete the reaction. The 337.0 g of distilled water was added and an internal temperature was cooled to 10-15° C. by using an ice bath, after which the resulting mixture was stirred for 12 hours and crystallized. The resulting crystals were filtered and the filtrate was washed with purified water. The obtained solid was vacuum dried at 40° C. to yield 117.2 g of 4-trifluoroacetamido-3-hydroxy-N,N-dimethyl-5-nitrobenzamide (yield: 86%).

¹H-NMR (400 MHZ, DMSO-d₆): 10.9 (s, 1H), 9.8 (s, 1H), 7.3 (d, 1H), 7.2 (d, 1H), 3.0 (s, 6H)

### Example 1-7. Preparation of 4-acetamido-3-hydroxy-N,N-dimethyl-5-nitrobenzamide

The 1034.8 g of purified water, 701.9 g of dimethyl sulfoxide and 144.4 g of potassium carbonate were sequentially added to a reactor. The resulting solution was stirred for 30 minutes while an internal temperature was raised to 37° C. The 3.8 g of copper (I) bromide, 4.6 g of 1,2-dimethylethylenediamine and 344.9 g of purified water were added and stirred while maintaining the internal temperature of 37° C. Then, a solution obtained by dissolving 172.5 g of 4-acetamido-3-bromo-N,N-dimethyl-5-nitrobenzamide in 38.5 g of dimethyl sulfoxide was added to the reactor. The reaction solution was stirred for 60 minutes while maintaining the same at 37° C., so as to complete the reaction. The 345.0 g of distilled water was added and an internal temperature was cooled to 10-15° C. by using an ice bath, after which the resulting mixture was stirred for 12 hours and crystallized. The resulting crystals were filtered and the filtrate was washed with purified water. The obtained solid was vacuum dried at 40° C. to yield 132.6 g of 4-acetamido-3-hydroxy-N,N-dimethyl-5-nitrobenzamide (yield: 95%).

¹H-NMR (400 MHZ, DMSO-d₆): 10.9 (s, 1H), 9.8 (s, 1H), 7.3 (d, 1H), 7.2 (d, 1H), 3.0 (s, 6H), 2.0 (s, 3H)

### Example 1-8. Preparation of 4-amino-3-hydroxy-N,N-dimethyl-5-nitrobenzamide

The 899.8 g of purified water, 610.3 g of dimethyl sulfoxide and 125.6 g of potassium carbonate were sequentially added to a reactor. The resulting solution was stirred for 30 minutes while an internal temperature was raised to 60° C. The 15.5 g of copper (I) bromide, 4.0 g of 1,2-dimethylethylenediamine and 299.9 g of purified water were added and stirred while maintaining the internal temperature of 60° C. Then, a solution obtained by dissolving 155.2 g of 4-amino-3-bromo-N,N-dimethyl-5-nitrobenzamide in 33.5 g of dimethyl sulfoxide was added to the reactor. The reaction solution was stirred for 10 minutes while maintaining the same at 60° C., so as to complete the reaction. The 300.0 g of distilled bath, after which the resulting mixture was stirred for 12 hours and crystallized. The crystals were filtered and the filtrate was washed with purified water. The obtained solid was vacuum dried at 40° C. to yield 117.7 g of 4-amino-3-hydroxy-N,N-dimethyl-5-nitrobenzamide (yield: 97%).

¹H-NMR (400 MHZ, DMSO-d₆): 10.7 (s, 1H), 7.6 (d, 1H), 7.1 (s, 2H), 7.0 (s, 1H), 3.0 (s, 6H)

### Example 1-9. Preparation of 4-trifluoroacetamido-3-hydroxy-N,N-dimethyl-5-nitrobenzamide

The 1010.8 g of purified water, 685.6 g of dimethyl sulfoxide and 141.1 g of potassium carbonate were sequentially added to a reactor. The resulting solution was stirred for 90 minutes while an internal temperature was raised to 45° C. The 3.7 g of copper (I) bromide, 4.5 g of 1,2-dimethylethylenediamine and 336.9 g of purified water were added and stirred while maintaining the internal temperature of 45° C. Then, a solution obtained by dissolving 163.0 g of 4-trifluoroacetamido-3-bromo-N,N-dimethyl-5-nitrobenzamide in 37.6 g of dimethyl sulfoxide was added to the reactor. The reaction solution was stirred for 60 minutes while maintaining the same at 45° C., so as to complete the reaction. The 337.0 g of distilled water was added and an internal temperature was cooled to 10-15° C. by using an ice bath, after which the resulting mixture was stirred for 12 hours and crystallized. The resulting crystals were filtered and the filtrate was washed with purified water. The obtained solid was vacuum dried at 40° C. to yield 122.7 g of 4-trifluoroacetamido-3-hydroxy-N,N-dimethyl-5-nitrobenzamide (yield: 90%).

¹H-NMR (400 MHZ, DMSO-d₆): 10.9 (s, 1H), 9.8 (s, 1H), 7.3 (d, 1H), 7.2 (d, 1H), 3.0 (s, 6H)

### Example 2. Step 2

### Example 2-1. Preparation of 4-hydroxy-N,N,2-trimethyl-1H-benzo[d]imidazol-6-carboxamide

The 994.8 g of methanol and 104.3 g of 4-amino-3-hydroxy-N,N-dimethyl-5-nitrobenzamide were sequentially added to a reactor. The resulting solution was stirred at 20-30° C. to dissolve all the solid therein, after which 2.6 g of 10% palladium carbon was added and an internal temperature was raised to 40° C. After replacing an inside of the reactor with hydrogen gas, the resulting mixture was stirred for 30 minutes under a hydrogen pressure of 0.2 MPa. After slowly cooling an internal temperature of the reactor to 20-30° C., 225.4 g of triethyl orthoacetate was added and stirred for 12 hours to complete the reaction. The reaction solution was concentrated under vacuum under 40° C. to remove methanol, and then 1564.5 g of purified water was added. The resulting mixture was stirred at 20-30° C. for 12 hours to ripen crystals and filter the same out, and then the filtrate was washed with purified water. The obtained solid was vacuum dried at 40° C. to yield 96.5 g of 4-hydroxy-N,N,2-trimethyl-1H-benzo[d]imidazol-6-carboxamide (yield: 95%).

¹H-NMR (400 MHZ, DMSO-d₆): 12.3, 12.2 (d, 1H), 10.0, 9.8 (d, 1H), 7.0, 6.9 (d, 1H), 6.6, 6.5 (d, 1H), 2.9 (s, 6H), 2.5 (s, 3H)

### Example 2-2. Preparation of 4-hydroxy-N,N,2-trimethyl-1H-benzo[d]imidazol-6-carboxamide

The 548.2 g of methanol, 138.4 g of 4-trifluoroacetamido-3-hydroxy-N,N-dimethyl-5-nitrobenzamide and 45.8 g of triethylamine were sequentially added to a reactor. The resulting solution was stirred at 20-30° C. to dissolve all the solid therein, after which 3.1 g of 10% palladium carbon was added and an internal temperature was raised to 40° C. After replacing an inside of the reactor with hydrogen gas, the resulting mixture was stirred for 30 minutes under a hydrogen pressure of 0.2 MPa. After slowly cooling an internal temperature of the reactor to 20-30° C., 185.9 g of triethyl orthoacetate was added and stirred for 12 hours to complete the reaction. The reaction solution was concentrated under vacuum under 40° C. to remove methanol, and then 1840.5 g of purified water was added. The resulting mixture was stirred at 20-30° C. for 12 hours to ripen crystals and filter the same out, and then the filtrate was washed with purified water. The obtained solid was vacuum dried at 40° C. to yield 73.7 g of 4-hydroxy-N,N,2-trimethyl-1H-benzo[d]imidazol-6-carboxamide (yield: 88%).

¹H-NMR (400 MHZ, DMSO-d₆): 12.3, 12.2 (d, 1H), 10.0, 9.8 (d, 1H), 7.0, 6.9 (d, 1H), 6.6, 6.5 (d, 1H), 2.9 (s, 6H), 2.5 (s, 3H)

### Example 2-3. Preparation of 4-hydroxy-N,N,2-trimethyl-1H-benzo[d]imidazol-6-carboxamide

The 1458.6 g of dimethyl sulfoxide, 1046.2 g of ethanol, 132.6 g of 4-acetamido-3-hydroxy-N,N-dimethyl-5-nitrobenzamide and 432.0 g of sodium dithionate were sequentially added to a reactor. The resulting solution was stirred for 24 hours while an internal temperature was raised to 90° C., so as to complete the reaction. The reaction solution was concentrated under vacuum under 40° C. to remove ethanol, and then 1989.0 g of purified water was added. The resulting mixture was stirred at 20-30° C. for 12 hours to ripen crystals and filter the same out, and then the filtrate was washed with purified water. The obtained solid was vacuum dried at 40° C. to yield 87.0 g of 4-hydroxy-N,N,2-trimethyl-1H-benzo[d]imidazol-6-carboxamide (yield: 80%).

¹H-NMR (400 MHZ, DMSO-d₆): 12.3, 12.2 (d, 1H), 10.0, 9.8 (d, 1H), 7.0, 6.9 (d, 1H), 6.6, 6.5 (d, 1H), 2.9 (s, 6H), 2.5 (s, 3H)

### Example 2-4. Preparation of 4-hydroxy-N,N,2-trimethyl-1H-benzo[d]imidazol-6-carboxamide

The 1387.7 g of acetic acid and 132.2 g of 4-amino-3-hydroxy-N,N-dimethyl-5-nitrobenzamide were sequentially added to a reactor. The 65.6 g of powder-type reduced iron was added while stirring at 20-30° C. and the resulting mixture was stirred for 12 hours while raising an internal temperature up to 70° C. After slowly cooling the internal temperature of the reactor to 20-30° C., the reaction solution was passed through celite filter paper, so as to remove residual solids. After that, 285.6 g of triethyl orthoacetate was added and stirred for 12 hours to complete the reaction. The 1982.5 g of purified water was added to the reaction solution, a pH of the solution was adjusted to 7-8, and the resulting solution was stirred at 20-30° C. for 12 hours, so as to ripen the crystals and filter the same out. Then, the filtrate was washed with purified water. The obtained solid was vacuum dried at 40° C. to yield 96.5 g of 4-hydroxy-N,N,2-trimethyl-1H-benzo[d]imidazol-6-carboxamide (yield: 75%).

¹H-NMR (400 MHZ, DMSO-d₆): 12.3, 12.2 (d, 1H), 10.0, 9.8 (d, 1H), 7.0, 6.9 (d, 1H), 6.6, 6.5 (d, 1H), 2.9 (s, 6H), 2.5 (s, 3H)

### Example 2-5. Preparation of 4-hydroxy-N,N,2-trimethyl-1H-benzo[d]imidazol-6-carboxamide

The 548.2 g of methanol, 138.4 g of 4-trifluoroacetamido-3-hydroxy-N,N-dimethyl-5-nitrobenzamide and 45.8 g of triethylamine were sequentially added to a reactor. The resulting solution was stirred at 20-30° C. for one hour and concentrated under vacuum under 40° C. so as to remove methanol. The 1453.6 g of acetic acid was added to the resulting concentrate, after which 48.1 g of powder-type reduced iron was added and the resulting mixture was stirred for 12 hours while raising an internal temperature up to 70° C. After slowly cooling the internal temperature of the reactor to 20-30° C., the reaction solution was passed through celite filter paper, so as to remove residual solids. After that, 129.5 g of triethyl orthoacetate was added and stirred for 12 hours to complete the reaction. The 2076.0 g of purified water was added to the reaction solution, a pH of the solution was adjusted to 7-8, and the resulting solution was stirred at 20-30° C. for 12 hours, so as to ripen the crystals and filter the same out. Then, the filtrate was washed with purified water. The obtained solid was vacuum dried at 40° C. to yield 73.7 g of 4-hydroxy-N,N,2-trimethyl-1H-benzo[d]imidazol-6-carboxamide (yield: 78%).

¹H-NMR (400 MHz, DMSO-d₆): 12.3, 12.2 (d, 1H), 10.0, 9.8 (d, 1H), 7.0, 6.9 (d, 1H), 6.6, 6.5 (d, 1H), 2.9 (s, 6H), 2.5 (s, 3H)

### Example 3. Step 3

### Example 3-1. Preparation of 6-(dimethylcarbamoyl)-2-methyl-1-tosyl-1H-benzo[d]imidazol-4-yl acetate

A mixed solution of 887.1 g of methylene chloride and 65.0 g of pyridine was prepared in a reactor. After cooling the solution to 3-5° C., 66.7 g of 4-hydroxy-N,N,2-trimethyl-1H-benzo[d]imidazol-6-carboxamide and 23.9 g of acetyl chloride were sequentially added while slowly stirring and then stirred at 3-5° C. for two hours. After observing that the solid was completely dissolved and re-precipitated in the reaction solution, a temperature of the reaction solution was raised to 20-30° C., and 42.0 g of potassium carbonate and 58.0 g of p-toluenesulfonyl chloride were sequentially added and further stirred for 30 minutes. An internal temperature was slowly cooled to 3-5° C. by using an ice bath, and 333.5 g of purified water was added to complete the reaction. The solution was stirred for one hour while raising the temperature to 20-30° C. An organic layer was separated and concentrated under vacuum under 40° C. to remove the reaction solvent, after which 157.3 g of acetonitrile and 200.1 g of purified water were added and stirred to crystallize. The resulting crystals were filtered and the filtrate was washed with a mixed solution of acetonitrile and purified water. The obtained solid was vacuum dried at 40° C. to yield 116.3 g of 6-(dimethylcarbamoyl)-2-methyl-1-tosyl-1H-benzo[d]imidazol-4-yl acetate (yield: 92%).

¹H-NMR (400 MHZ, DMSO-d₆): 8.0, 7.9 (d, 2H), 7.5, 7.4 (d, 2H), 7.3 (s, 1H), 6.7 (s, 1H), 3.0, 2.9 (d, 6H), 2.8 (s, 3H), 2.4 (s, 3H), 2.0 (s, 3H)

### Example 3-2. Preparation of 6-(dimethylcarbamoyl)-2-methyl-1-((trifluoromethyl) sulfonyl)-1H-benzo[d]imidazol-4-yl acetate

A mixed solution of 691.6 g of methylene chloride and 37.5 g of pyridine was prepared in a reactor. The 52.0 g of 4-hydroxy-N,N,2-trimethyl-1H-benzo[d]imidazol-6-carboxamide and 18.6 g of acetyl chloride were sequentially added while slowly stirring the solution to 20-30° C., and then stirred at 20-30° C. for one hour. After observing that the solid was completely dissolved and re-precipitated in the reaction solution, 32.8 g of potassium carbonate and 40.0 g of triflyl chloride were sequentially added and further stirred for three hours. An internal temperature was slowly cooled to 3-5° C. by using an ice bath, and 260.0 g of purified water was added to complete the reaction. The solution was stirred for one hour while raising the temperature to 20-30° C. An organic layer was separated and concentrated under vacuum under 40° C. to remove the reaction solvent, after which 312.0 g of purified water was added and stirred to crystallize. The resulting crystals were filtered and the filtrate was washed with methanol. The obtained solid was vacuum dried at 40° C. to yield 76.5 g of 6-(dimethylcarbamoyl)-2-methyl-1-((trifluoromethyl) sulfonyl)-1H-benzo[d]imidazol-4-yl acetate (yield: 82%).

¹H-NMR (400 MHZ, DMSO-d₆): 7.3 (s, 1H), 6.7 (s, 1H), 3.0, 2.9 (d, 6H), 2.8 (s, 3H), 2.0 (s, 3H)

### Example 3-3. Preparation of 6-(dimethylcarbamoyl)-2-methyl-1-(methylsulfonyl)-1H-benzo[d]imidazol-4-yl acetate

A mixed solution of 980.2 g of methylene chloride and 26.6 g of pyridine was prepared in a reactor. After warming the solution up to 35-40° C., 73.7 g of 4-hydroxy-N,N,2-trimethyl-1H-benzo[d]imidazol-6-carboxamide and 26.4 g of acetyl chloride were sequentially added while slowly stirring and then stirred at 40° C. for 30 minutes. After observing that the solid was completely dissolved and re-precipitated in the reaction solution, a temperature of the solution was cooled to 20-30° C., and 46.5 g of potassium carbonate and 38.5 g of methanesulfonyl chloride were sequentially added and further stirred for three hours. An internal temperature was slowly cooled to 3-5° C. by using an ice bath, and 368.5 g of purified water was added to complete the reaction. The solution was stirred for one hour while raising the temperature to 20-30° C. An organic layer was separated and concentrated under vacuum under 40° C. to remove the reaction solvent, after which 291.9 g of methanol was added and stirred to crystallize. The resulting crystals were filtered and the filtrate was washed with methanol. The obtained solid was vacuum dried at 40° C. to yield 91.3 g of 6-(dimethylcarbamoyl)-2-methyl-1-(methylsulfonyl)-1H-benzo[d]imidazol-4-yl acetate (yield: 80%).

¹H-NMR (400 MHZ, DMSO-d₆): 7.3 (s, 1H), 6.7 (s, 1H), 3.0, 2.9 (d, 6H), 2.8 (s, 3H), 2.4 (s, 3H), 2.0 (s, 3H)

### Example 4. Step 4

### Example 4-1. Preparation of 4-hydroxy-N,N,2-trimethyl-1-tosyl-1H-benzo[d]imidazol-6-carboxamide

A mixed solution of 213.8 g of methanol and 270.0 g of purified water was prepared in a reactor. The 90.0 g of 6-(dimethylcarbamoyl)-2-methyl-1-tosyl-1H-benzo[d]imidazol-4-yl acetate was added while slowly stirring the solution at 20-30° C. An internal temperature was slowly cooled to 3-5° C. by using an ice bath, and a pH of the reaction solution was adjusted to 10.0-10.2 by using 30-35% aqueous ammonia. The reaction solution was further stirred at 3-5° C. for 16 hours while maintaining the pH thereof. The resulting crystals were filtered and washed with a mixed solution of methanol and purified water. The obtained solid was vacuum dried at 40° C. to yield 76.8 g of 4-hydroxy-N,N,2-trimethyl-1-tosyl-1H-benzo[d]imidazol-6-carboxamide (yield: 95%).

¹H-NMR (400 MHZ, DMSO-d₆): 10.4 (s, 1H), 8.0, 7.9 (d, 2H), 7.5, 7.4 (d, 2H), 7.3 (s, 1H), 6.7 (s, 1H), 3.0, 2.9 (d, 6H), 2.8 (s, 3H), 2.4 (s, 3H)

### Example 4-2. Preparation of 4-hydroxy-N,N,2-trimethyl-1-((trifluoromethyl) sulfonyl)-1H-benzo[d]imidazol-6-carboxamide

A mixed solution of 130.8 g of methanol and 165.1 g of purified water was prepared in a reactor. The 55.0 g of 6-(dimethylcarbamoyl)-2-methyl-1-((trifluoromethyl) sulfonyl)-1H-benzo[d]imidazol-4-yl acetate was added while slowly stirring the solution at 20-30° C. An internal temperature was slowly cooled to 3-5° C. by using an ice bath, and a pH of the reaction solution was adjusted to 8.0-8.2 by using 30-35% aqueous ammonia. After further adding 330.2 g of purified water, the reaction solution was further stirred at 3-5° C. for 16 hours while maintaining the pH thereof. The resulting crystals were filtered and washed with a mixed solution of methanol and purified water. The obtained solid was vacuum dried at 40° C. to yield 46.2 g of 4-hydroxy-N,N,2-trimethyl-1-((trifluoromethyl) sulfonyl)-1H-benzo[d]imidazol-6-carb oxamide (yield: 94%).

¹H-NMR (400 MHZ, DMSO-d₆): 10.4 (s, 1H), 7.3 (s, 1H), 6.7 (s, 1H), 3.0, 2.9 (d, 6H), 2.8 (s, 3H)

### Example 4-3. Preparation of 4-hydroxy-N,N,2-trimethyl-1-(methylsulfonyl)-1H-benzo[d]imidazol-6-carboxamide

A mixed solution of 171.1 g of methanol and 216.0 g of purified water was prepared in a reactor. The 72.0 g of 6-(dimethylcarbamoyl)-2-methyl-1-(methylsulfonyl)-1H-benzo[d]imidazol-4-yl acetate was added while slowly stirring the solution at 20-30° C. An internal temperature was slowly cooled to 3-5° C. by using an ice bath, and a pH of the reaction solution was adjusted to 10.0-10.5 by using 30-35% aqueous ammonia. After further adding 216.0 g of purified water, the reaction solution was further stirred at 3-5° C. for 16 hours while maintaining the pH thereof. The resulting crystals were filtered and washed with a mixed solution of methanol and purified water. The obtained solid was vacuum dried at 40° C. to yield 59.3 g of 4-hydroxy-N,N,2-trimethyl-1-(methylsulfonyl)-1H-benzo[d]imidazol-6-carboxyamide (yield: 94%).

¹H-NMR (400 MHZ, DMSO-d₆): 10.4 (s, 1H), 7.3 (s, 1H), 6.7 (s, 1H), 3.0, 2.9 (d, 6H), 2.8 (s, 3H), 2.4 (s, 3H)

### Example 5. Step 3)+Step 4) (In Situ)

### Example 5-1. Preparation of 4-hydroxy-N,N,2-trimethyl-1-tosyl-1H-benzo[d]imidazol-6-carboxamide

A mixed solution of 663.7 g of methylene chloride and 48.7 g of pyridine was prepared in a reactor. After cooling the solution to 3-5° C., 50.0 g of 4-hydroxy-N,N,2-trimethyl-1H-benzo[d]imidazol-6-carboxamide and 17.9 g of acetyl chloride were sequentially added while slowly stirring and then stirred at 3-5° C. for two hours. After observing that the solid was completely dissolved and re-precipitated in the reaction solution, a temperature of the reaction solution was raised to 20-30° C., and 31.5 g of potassium carbonate and 87.0 g of p-toluenesulfonyl chloride were sequentially added and further stirred for 30 minutes. An internal temperature was slowly cooled to 3-5° C. by using an ice bath, and 50.0 g of purified water and 7.4 g of tetra-n-butylammonium bromide were added thereto. While vigorously stirring the reaction solution, 157.6 g of potassium carbonate was added and the reaction was continued for 16 hours. The reaction was completed by adding 250.0 g of purified water and adjusting a pH of the reaction solution to 5-7. The solution was stirred for one hour while raising the temperature to 20-30° C. An organic layer was separated and concentrated under vacuum under 40° C. to remove the reaction solvent, after which 118.8 g of methanol and 150.0 g of purified water were added and stirred to crystallize. The resulting crystals were filtered and the filtrate was washed with a mixed solution of methanol and purified water. The obtained solid was vacuum dried at 40° C. to yield 76.7 g of 4-hydroxy-N,N,2-trimethyl-1-tosyl-1H-benzo[d]imidazol-6-carboxamide (yield: 90%).

¹H-NMR (400 MHZ, DMSO-d₆): 10.4 (s, 1H), 8.0, 7.9 (d, 2H), 7.5, 7.4 (d, 2H), 7.3 (s, 1H), 6.7 (s, 1H), 3.0, 2.9 (d, 6H), 2.8 (s, 3H), 2.4 (s, 3H)

### Example 5-2. Preparation of 4-hydroxy-N,N,2-trimethyl-1-((trifluoromethyl) sulfonyl)-1H-benzo[d]imidazol-6-carboxamide

A mixed solution of 663.6 g of methylene chloride and 36.1 g of pyridine was prepared in a reactor. The 50.0 g of 4-hydroxy-N,N,2-trimethyl-1H-benzo[d]imidazol-6-carboxamide and 17.9 g of acetyl chloride were sequentially added while slowly stirring the solution to 20-30° C., and then stirred at 20-30° C. for one hour. After observing that the solid was completely dissolved and re-precipitated in the reaction solution, 31.5 g of potassium carbonate and 76.9 g of triflyl chloride were sequentially added and further stirred for three hours. An internal temperature was slowly cooled to 3-5° C. by using an ice bath, and 50.0 g of purified water and 7.4 g of tetra-n-butylammonium bromide were added thereto. While vigorously stirring the reaction solution, 44.0 g of 30-35% aqueous ammonia was added and the reaction was continued for 16 hours. The reaction was completed by adding 250.0 g of purified water and adjusting a pH of the reaction solution to 5-7. The solution was stirred for one hour while raising the temperature to 20-30° C. An organic layer was separated and concentrated under vacuum under 40° C. to remove the reaction solvent, after which 118.8 g of methanol and 450.0 g of purified water were added and stirred to crystallize. The resulting crystals were filtered and the filtrate was washed with a mixed solution of methanol and purified water. The obtained solid was vacuum dried at 40° C. to yield 64.1 g of 4-hydroxy-N,N,2-trimethyl-1-((trifluoromethyl) sulfonyl)-1H-benzo[d]imidazol-6-carb oxamide (yield: 80%).

¹H-NMR (400 MHZ, DMSO-d₆): 10.4 (s, 1H), 7.3 (s, 1H), 6.7 (s, 1H), 3.0, 2.9 (d, 6H), 2.8 (s, 3H)

### Example 5-3. Preparation of 4-hydroxy-N,N,2-trimethyl-1-(methylsulfonyl)-1H-benzo[d]imidazol-6-carboxamide

A mixed solution of 663.5 g of methylene chloride and 18.0 g of pyridine was prepared in a reactor. After warming the solution up to 35-40° C., 50.0 g of 4-hydroxy-N,N,2-trimethyl-1H-benzo[d]imidazol-6-carboxamide and 17.9 g of acetyl chloride were sequentially added while slowly stirring and then stirred at 40° C. for 30 minutes. After observing that the solid was completely dissolved and re-precipitated in the reaction solution, a temperature of the solution was cooled to 20-30° C., and 31.5 g of potassium carbonate and 52.2 g of methanesulfonyl chloride were sequentially added and further stirred for three hours. An internal temperature was slowly cooled to 3-5° C. by using an ice bath, and 50.0 g of purified water and 7.4 g of tetra-n-butylammonium bromide were added thereto. While vigorously stirring the reaction solution, 88.0 g of 30-35% aqueous ammonia was added and the reaction was continued for 16 hours. The reaction was completed by adding 250.0 g of purified water and adjusting a pH of the reaction solution to 5-7. The solution was stirred for one hour while raising the temperature to 20-30° C. An organic layer was separated and concentrated under vacuum under 40° C. to remove the reaction solvent, after which 118.8 g of methanol and 300.0 g of purified water were added and stirred to crystallize. The resulting crystals were filtered and the filtrate was washed with a mixed solution of methanol and purified water. The obtained solid was vacuum dried at 40° C. to yield 53.6 g of 4-hydroxy-N,N,2-trimethyl-1-(methylsulfonyl)-1H-benzo[d]imidazol-6-carboxamide (yield: 80%).

¹H-NMR (400 MHZ, DMSO-d₆): 10.4 (s, 1H), 7.3 (s, 1H), 6.7 (s, 1H), 3.0, 2.9 (d, 6H), 2.8 (s, 3H), 2.4 (s, 3H)

While specific portions of the present invention have been described in detail above, it is apparent to those skilled in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. A method for preparing benzimidazole derivatives, the method comprising:
1) preparing a compound represented by formula 2 below by reacting a compound represented by formula 3 below with a monovalent copper catalyst in a presence of a ligand represented by formula 4 below or a stereoisomer thereof;
2) preparing a compound represented by formula 5 below by reacting a compound represented by formula 2 below; and
3) preparing a compound represented by formula 1 below by reacting a compound represented by formula 5 below, acetyl chloride, and a compound represented by formula 6 below in a presence of a base, 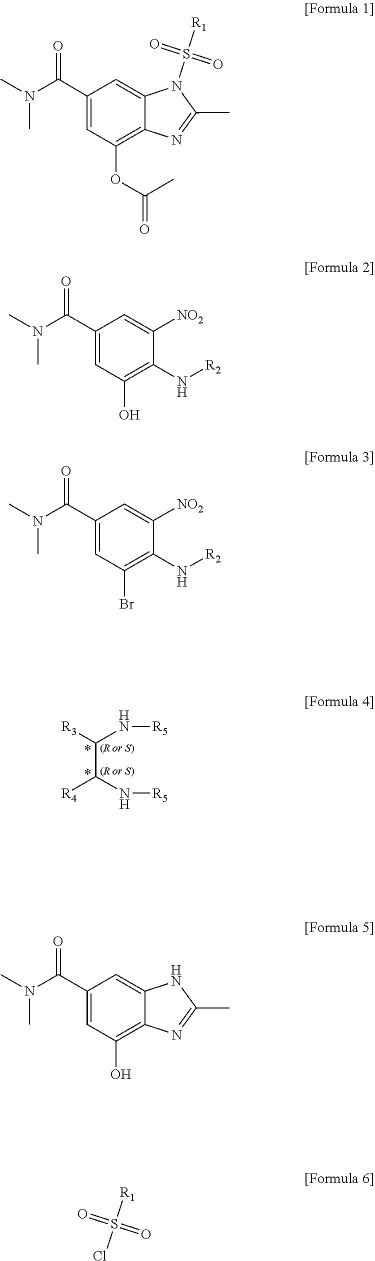
wherein,
R₁ is C₁₋₄ alkyl or phenyl, wherein at least one H of the C₁₋₄ alkyl and phenyl may be substituted with halogen or C₁₋₄ alkyl,
R₂ is H, C₁₋₄ alkyl or acetyl, wherein at least one H of the acetyl may be substituted with halogen,
R₃ and R₄ are each independently selected from the group consisting of H, C₁₋₄ alkyl and C₃₋₇ cycloalkyl, or together form a 3- to 7-membered alicyclic ring, wherein at least one H of the C₁₋₄ alkyl, C₃₋₇ cycloalkyl and the formed 3- to 7-membered alicyclic ring may be substituted with halogen, and
R₅ is H, C₁₋₄ alkyl or acetyl.

2. The method of claim 1, wherein,
R₁ is methyl, trifluoromethyl or tolyl,
R₂ is H, acetyl or trifluoroacetyl,
R₃ and R₄ are methyl, or together form a 6-membered alicyclic ring, and
R₅ is H or methyl.

3. The method of claim 1, further comprising:
4) preparing a compound represented by formula 1-1 below by adjusting a pH of the compound represented by above formula 1: 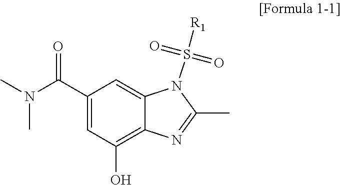
wherein,
R₁ is the same as in above formula I of claim 1.

4. The method of claim 1, wherein R₁ is methyl, trifluoromethyl or tolyl.

5. The method of claim 1, wherein the monovalent copper catalyst of above 1) is one selected from the group consisting of cuprous chloride, copper bromide, copper iodide and cuprous oxide.

6. The method of claim 1, wherein a molar ratio of the compound represented by above formula 3, the monovalent copper catalyst, and the ligand represented by above formula 4 or the stereoisomer thereof in above 1) is 10:2:4 to 10:0.5:0.5.

7. The method of claim 1, wherein above 1) is performed in at least one solvent selected from the group consisting of water, acetonitrile, tetrahydrofuran, dimethylsulfoxide, dimethylformamide and 1,4-dioxane.

8. The method of claim 1, wherein above 1) is performed by adjusting a temperature between 37° C. and 60° C. and stirring for 10 to 60 minutes.

9. The method of claim 1, wherein above 2) is to prepare the compound represented by above formula 5 by subjecting the compound represented by above formula 2 to a reductive cyclization reaction.

10. The method of claim 9, wherein the reductive cyclization of above 2) is performed by adding a reducing agent or a reducing agent and an acetyl source.

11. The method of claim 10, wherein the reducing agent is at least one selected from the group consisting of H₂/10% palladium carbon, reduced iron and sodium dithionate.

12. The method of claim 10, wherein the acetyl source is at least one selected from the group consisting of triethyl orthoacetate and acetyl acetone.

13. The method of claim 1, wherein the base of above 3) is at least one selected from the group consisting of potassium carbonate, sodium carbonate, cesium carbonate, aqueous ammonia and pyridine.

14. The method of claim 1, wherein a molar ratio of the compound represented by above formula 5 and the compound represented by above formula 6 in above 3) is 1:1 to 1:2.5.

15. The method of claim 1, wherein a molar ratio of the acetyl chloride and the base in above 3) is 1:2 to 1:4.

16. The method of claim 1, wherein a molar ratio of the compound represented by above formula 5 and the acetyl chloride in above 3) is 1:1 to 1:1.5.

17. The method of claim 1, wherein a molar ratio of the compound represented by above formula 6 and the base in above 3) is 1:1 to 1:4.

18. The method of claim 1, wherein above 3) is performed in at least one solvent selected from the group consisting of acetone, methyl ethyl ketone, ethyl acetate, methylene chloride, chloroform and acetonitrile.

19. The method of claim 18, wherein above 3) comprises:
a) adding the compound represented by above formula 5 and acetyl chloride to a solvent; and
b) adding the compound represented by above formula 6 in the presence of a base.

20. The method of claim 19, wherein the compound represented by above formula 5 in above a) is added in an amount of 5% (w/v) to 33% (w/v) relative to the solvent.

21. The method of claim 19, wherein above a) is performed by adjusting a temperature between 3° C. and 40° C. and stirring for 30 to 120 minutes.

22. The method of claim 19, wherein above b) is performed by adjusting a temperature between 27° C. and 40° C. and stirring for 10 to 180 minutes.

23. The method of claim 3, wherein above 4) is to adjust the pH between 8 and 12.

24. The method of claim 3, wherein above 4) is performed by adjusting a temperature between 3° C. and 10° C.

25. The method of claim 3, wherein above 3) and 4) are performed in situ.

26. A compound represented by formula 1 below: 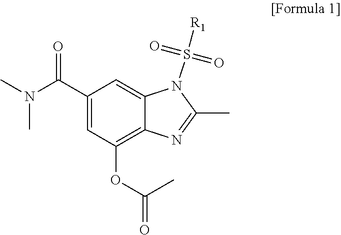
wherein,
R₁ is C₁₋₄ alkyl or phenyl, wherein at least one H of the C₁₋₄ alkyl and phenyl may be substituted with halogen or C₁₋₄ alkyl.

27. The compound of claim 26, wherein R₁ is methyl, trifluoromethyl or tolyl.

28. The compound of claim 26, wherein the compound represented by above formula 1 is selected from the group consisting of:
6-(dimethylcarbamoyl)-2-methyl-1-tosyl-1H-benzo[d]imidazol-4-yl acetate;
6-(dimethylcarbamoyl)-2-methyl-1-((trifluoromethyl) sulfonyl)-1H-benzo[d]imidazol-4-yl acetate; and
6-(dimethylcarbamoyl)-2-methyl-1-methylsulfonyl-1H-benzo[d]imidazol-4-yl acetate.
